(19) United States
(12) Patent Application Publication
(10) Pub. No.: US 2022/0226526 A1
(43) Pub. Date: Jul. 21, 2022

(54) Portable sanitizing mat

(71) Applicant: Kevin Shafer, Carlsbad, NM (US)

(72) Inventor: Kevin Shafer, Carlsbad, NM (US)

(21) Appl. No.: 17/153,734

(22) Filed: Jan. 20, 2021

(51) Int. Cl. A61L 2/26; A61L 2/10

(52) U.S. Cl. CPC A61L 2/26; A61L 2202/11; A61L 2/10

(57) ABSTRACT

The present invention is directed to a mat for enclosed spaces which has both a disinfecting and illuminating function. The mat is foldable or collapsible for stowing purposes. The inner surface includes at least a pair of UV lamps or light strips positioned thereon. The lamps, which may use UV-C radiation, are preferably powered by the local AC power. When disinfection is desired, the mat may be deployed within the enclosed space on any suitable surface and electrically connected to the AC power source. The UV lamps give off radiation which disinfects the enclosed space while unoccupied.

### BACKGROUND OF THE INVENTION

### 1. Field of the Invention

This invention relates to sanitizing apparatuses. More particularly, it relates to a mat for that can be used to disinfect and illuminate the interior of an enclosed space.

### 2. Description of the Prior Art

In light of the Covid-19 outbreak it has become increasingly important to disinfect all commonly used surfaces, especially interior surfaces, as well as commonly occupied areas such as bathrooms. The usually enclosed tight confines of e.g., a bathroom or a vehicle interior provides a perfect environment for the spread of pathogens. The problem is exacerbated when people and items, e.g., packages, are constantly entering an enclosed space and potentially depositing pathogens therein. Packages from a high traffic area like the interior of grocery stores have a good probability of depositing pathogens wherever they are stored or transported. Moreover, aerosolized pathogens produced by, e.g., coughing or sneezing can remain in an area for some time after they are emitted. Accordingly, methods and apparatuses have been devised to deal with this problem.

United States Patent Application 20170100989 discloses methods and systems for reducing microbial contamination and growth in an automotive HVAC system by irradiating one or more air contacting surfaces of the HVAC system with antimicrobial UV-C light. This type of application is very environmentally specific and cannot be adapted to any other use. Moreover, it is not in any way portable.

While these and other prior art methods are generally effective for their intended purposes, they are singular in their utility.

The present invention is directed to a disinfecting mat useful for disinfecting the interiors of or specific targeted surfaces within enclosures which has both a disinfecting and illuminating function. The mat may be positioned on any surface, or may optionally be used in a free standing mode and includes an arrangement of UV-C lamps or strips of UV-C emitting LEDs. The mat is foldable or rollable for stowing purposes. The UV lighting is preferably primarily powered by a home or mobile electrical system, and gives off radiation which disinfects the targeted area while unoccupied. The mats, which are lightweight, may be used in any enclosed space needing disinfection.

### SUMMARY OF THE INVENTION

It is a major object of the invention to provide a disinfecting mat assembly.

It is another object of the invention to provide an improved mat that can disinfect an enclosed space such as a vehicle interior.

It is another object of the invention to provide an improved automotive mat that uses UV lighting to disinfect.

It is another object of the invention to provide an improved disinfecting mat that can be powered by a local electrical system.

### BRIEF DESCRIPTION OF THE DRAWINGS

FIG. 1 shows a perspective view of a mat assembly formed in accordance with the invention.

FIG. 2 shows a side view of a mat assembly formed in accordance with the invention positioned on a surface in an enclosure.

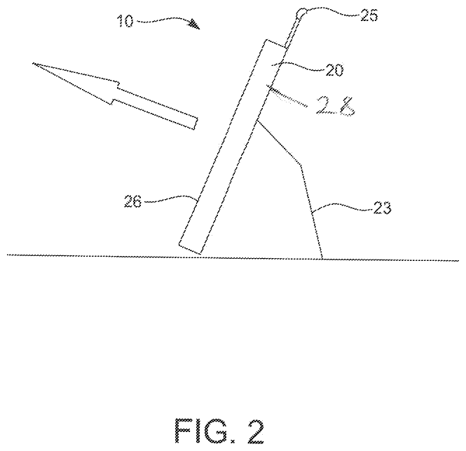

FIG. 3 shows a diagram illustrating the electrical connections of the mat assembly.

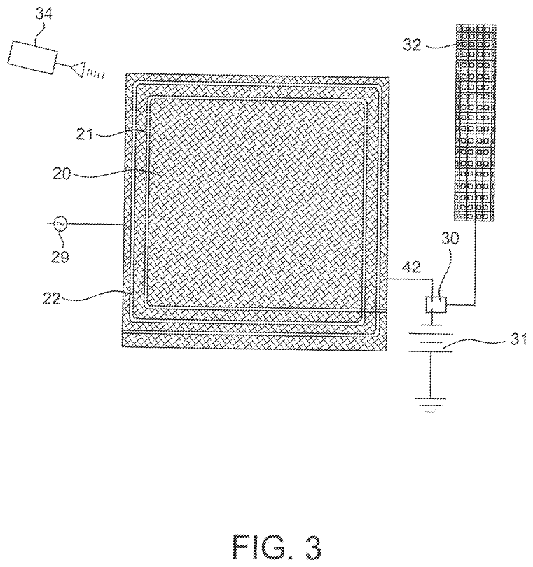

### DETAILED DESCRIPTION OF THE PREFERRED EMBODIMENTS

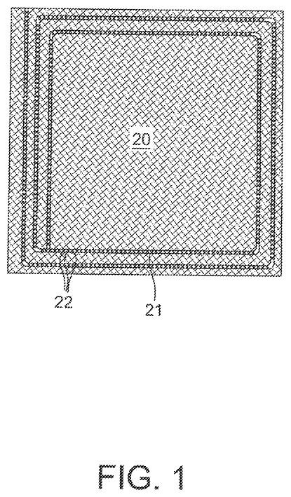

Referring now to the drawings, FIG. 1 illustrates the inventive sanitizing mat assembly, generally indicated by the numeral 10. The assembly 10 has the ability to disinfect, using UV radiation, a good part of the interior of an enclosed space. The assembly 10 also provides additional interior lighting, the lighting having a decorative aspect.

Ultraviolet germicidal irradiation (UVGI) is a disinfection method that uses short-wavelength ultraviolet (UV-C) light to kill or inactivate microorganisms. One mechanism by which UV-C deactivates microorganisms is by destroying nucleic acids and disrupting their DNA, leaving them unable to perform vital cellular functions. The administration of UV-C radiation is becoming widely adopted by many hospitals as a more effective and reliable means of surface disinfection, as compared to the use of chemical cleaning agents alone. The effectiveness of germicidal UV-C irradiation depends on factors such as the length of time a microorganism is exposed to UV-C, the intensity and wavelength of the UV-C radiation, the presence of particles that can protect the microorganisms from UV, and a microorganism's ability to withstand UV-C during its exposure. In air and surface disinfection applications, a UV-C radiation source will disinfect the air and surfaces of an interior space which receives the radiation. In light of the Covid-19 pandemic, there is now a great need to disinfect all areas and surfaces that are exposed to people (or objects), especially in an enclosed environment like a vehicle interior.

Accordingly, the present invention is directed to an apparatus for sanitizing and disinfecting an enclosed space such, which apparatus functions to provide both a disinfectant and a uniquely aesthetic auxiliary interior light arrangement. The apparatus preferably uses UV-C, but may also use UV-B or UV-A ultraviolet radiation.

The main body of the apparatus 10 is a mat 20 which is foldable or collapsible for stowage. The collapsible or rollable mat 20 which may be deployed in a bathroom, vehicle or other enclosed space, has a lateral and longitudinal dimension when in the extended state. The mat 20 may be sufficiently flexible to be rolled into a tube or otherwise compacted to fit in a small space. The mat 20 would also come in a variety of sizes varying from several inches to several feet in length so that at least some of the mats 20 are small enough to be portable or carried on a person. For example, an 8 inch mat 20 may be rolled into a tube and stored in a pocketbook. Cardboard covered with reflective bubble wrap insulation, or other similar lightweight materials are preferably used. A reflective material will enhance emission of radiation as would be apparent to one of skill in the art. The mat 20 may, e.g., be optionally made of a rubber or plastic webbing material to provide flexibility. The mat 20 has two sides, an upper outward facing side 26, and lower or underside 28. The mat 20 is designed to be as lightweight as possible to enhance portability regardless of material.

A number of features are included with the mat 20 to facilitate positioning in a number of areas. These positioning means as described below, allow for the mat 20 to be positioned as needed to emit radiation in the most effective pattern. For mats of all sizes, a collapsible stand such as a hinged flap 23 or like assembly is used to allow the mat 20 to be positioned in an upright position to irradiate a wall for example. Also, as the mat 20 is very lightweight, it can be hung from a wall or other vertical surface, with one or more apertured straps 25 attached to the mat 20 serving as an attachment means. The strap 25 may be secured to the wall using a thumbtack or other small non destructive fastening means so that the mat 20 may be positioned to irradiate a room or a nearby object. Both the strap 25 and flap 23 can be attached to the underside 28 of the mats 20 by Velcro or equivalent means. Mats 20 can be placed on a ceiling as by thumbtacks, to irradiate downwardly. As the mats are lightweight and thin, they may be hung without fear of injury or damage if they are dislodged. In short, mat 20 can be free standing or attached to virtually any surface in order to irradiate a desired area or object. Mats 20 maybe connected in series or parallel to the same power source 29 in the same manner as strings of decorative lights, with each mat 20 having circuitry for determining if sufficient power is available for operation as will be explained in more detail later.

The UV illumination is provided by a strip 21 of individual LED UV lights 22 of the kind commonly commercially available. The lights 22 are preferably UV-C, but may be UV-B or UV-C, and are positioned about the edges of the mat 20 by adhesive or the like. There may be two or three (or more) strips 21about the edges of the mat 20 as shown in FIG. 1, this to increase the intensity and dispersion of the radiated illumination. The perimeter positioning of the strips allows illumination from the lights 22 to be as widely dispersed as possible for a given size of the mat 20.

Power for the strip 21 may be supplied primarily by the local AC or DC source, as LED UV strips can be powered by AC or DC. If used in a structure such as the home or office, the local AC source 29 may be used. The mat 20 may include a built in or detachable battery 31 that is preferably rechargeable. A vehicle battery 31 may be used when the mat 20 is positioned within a vehicle. To that end, the lights 22 are connected to the vehicle auxiliary power outlet via wire 42. The wire 42 may also be connected to a USB outlet or other power source. If used in an enclosed space having 120V AC power, the strip 21 may be connected to an outlet via a power converter.

As UV lamps give off very bright and unusually colored light, the lights 22 may also be used to present an aesthetically pleasing interior lighting option, especially when used in a vehicle. The lights may be seen from a distance and produce a striking visual.

In order to prevent excessive battery 31 drain, circuitry 30 is provided to automatically shut the strip 21 down after a predetermined amount of time, typically about 30 minutes. The circuitry 30 is in the form of a microprocessor capable of receiving inputs from sensors and switching power to the strip 21.

A solar panel 32 may be provided with the assembly of the invention, the solar panel detachable from the assembly 10 so as to be stored when not in use. The solar panel 32 is electrically connected to circuitry 30, the circuitry determining if there is sufficient power available to power the lights 22. Circuitry 30 can also condition and regulate the power output from the solar panel 32.

Circuitry 30 may also include a sensor for detecting a signal from a transmitting fob 34, this can be the only on switch for safety purposes. The fob 34 is like that used for a keyless entry system, and transmits a low level activation signal which triggers a switch in the circuitry 30 which activates the strip 21. In this manner, the user is never accidentally irradiated while the strip 21 is active. Alternatively, a smartphone application can be downloaded, the application allowing the user to transmit a code for activating the strip 21 using a smartphone. It should be appreciated that as circuitry 30 is connected by wire 42 to the mat 20 it can be extended some distance from the mat 20 to receive a signal from the fob 34. Thus, if the mat 20 is disposed in a vehicle, the circuitry 30 and sensor can be positioned near a window.

In use, the user can position the mat 20 (or mats) as needed within an enclosure. One or more mats 20 would be positioned for the most effective treatment. For example, if used to disinfect a vehicle after a number of passengers have exited, it would be desirable to treat for aerosolized pathogens so that mats 20 positioned on the seats and ceiling would be most effective, as opposed to the vehicle floor. If a number of packages have been resting on the seats, a ceiling mat placement would be most effective. The mats may have a built in battery 31 as explained above, or be plugged into an AC or DC source. Thus, one or more mats 20, which mats can be carried in luggage, may be carried by a user to disinfect e.g., a hotel bathroom by plugging into the local AC source.

It is to be understood that the present invention is not limited to the sole embodiment described above, but encompasses any and all embodiments within the scope of the following claims:

I claim:

1. A disinfecting mat assembly for an enclosed space comprising:
a flat planar main body having an upper and lower side,
at least one strip of UV lights positioned on edge regions of said main body;
said UV lights electrically connected to a source of electrical power.

2. The assembly of claim 1 wherein said source of power is an AC power outlet.

3. The assembly of claim 1 wherein said source of power is an attached battery, said battery attached on or within said flat planar body.

4. The assembly of claim 3 wherein said source of power is a solar panel.

5. The assembly of claim 1 including a processor for controlling application of power from said source of power to said at least one UV light strip.

6. The assembly of claim 4 wherein said solar panel is electrically connected to said processor.

7. The assembly of claim 6 wherein said processor determines power output from said solar panel.

8. The assembly of claim 1 wherein solar panel is electrically connected to said battery for charging said battery.

9. The assembly of claim 1 wherein said main body is covered with a reflective bubble wrap material.

10. The assembly of claim 1 wherein said main body is made of a lightweight material.

11. The assembly of claim 10 wherein said lightweight material is cardboard.

12. The assembly of claim 1 wherein said light strip emits UVA, UVB, or UVC radiation.

13. The assembly of claim 1 wherein said main body includes a collapsible stand and an apertured strap to allow for positioning said main body within an enclosure.
